(19) United States
(12) Patent Application Publication
(10) Pub. No.: US 2025/0082185 A1
(43) Pub. Date: Mar. 13, 2025

(54) ENDOSCOPE IMAGING DEVICE AND ENDOSCOPE

(71) Applicant: FUJIFILM Corporation, Tokyo (JP)

(72) Inventors: Takuro ASAOKA, Kanagawa (JP); Takashi YASHIRO, Kanagawa (JP)

(73) Assignee: FUJIFILM Corporation, Tokyo (JP)

(21) Appl. No.: 18/767,920

(22) Filed: Jul. 9, 2024

(30) Foreign Application Priority Data
Sep. 8, 2023 (JP) 2023-146241

(51) Int. Cl. A61B 1/00

(52) U.S. Cl. CPC A61B 1/00188; A61B 1/00096

(57) ABSTRACT

Provided are an endoscope imaging device and an endoscope capable of adjusting a position of a fixed lens along an optical axis.

An endoscope imaging device includes: at least one movement group including a movable lens that is movable along an optical axis; a plurality of fixed groups including a fixed lens whose position is fixed with respect to the optical axis, and at least one of which is an adjustment group; a sensor that images an observation target; a lens drive portion that moves the movement group along the optical axis. The adjustment group includes a cylindrical holding part, and the holding part includes a grip part provided at one end part of the holding part and an end surface provided at the other end part opposite to the one end part in the optical axis direction. The grip part includes a projecting portion or a recessed portion whose diameter changes in a radial direction of the holding part, and the adjustment group is configured such that the end surface provided at the other end part of the holding part is disposed at a position spaced apart from the lens barrel body provided with a movement group, the fixed group, and the lens drive portion, or the fixed group in the optical axis direction, and the grip part is disposed at a position spaced apart from the lens barrel body or the fixed group in the optical axis direction and protrudes from the lens barrel body or the fixed group, and a side surface of the holding part is fixed to the lens barrel body or the fixed group.

### CROSS-REFERENCE TO RELATED APPLICATIONS

The present application claims priority under 35 U.S.C. § 119 to Japanese Patent Application No. 2023-146241, filed on Sep. 8, 2023. The above application is hereby expressly incorporated by reference, in its entirety, into the present application.

### BACKGROUND OF THE INVENTION

### 1. Field of the Invention

The present invention relates to an endoscope imaging device that acquires an image of an observation target and an endoscope, and particularly, to an endoscope imaging device that includes a movable lens that is movable along an optical axis and a fixed lens whose position is fixed with respect to the optical axis and an endoscope.

### 2. Description of the Related Art

Diagnosis and the like using an endoscope system that comprises an endoscope light source device, an endoscope (endoscope scope), and a processor device have been widely performed.

An insertion part that is inserted into a body of a subject to be examined is provided, and illumination light from the endoscope light source device passes through the insertion part and is emitted to an observation target. The endoscope images the observation target irradiated with illumination light by using an imaging element to generate an image signal. The processor device performs image processing on the image signal generated by the endoscope to generate an observation image to be displayed on a monitor. The imaging element is electrically connected to a signal cable via a circuit board configured with a flexible wiring board or the like, and the signal cable is electrically connected to the processor device. In recent years, an endoscope provided with an optical zooming function or a focusing function has been proposed.

For example, JP2008-110061A describes an endoscope that includes an imaging unit that is inserted into a distal end portion and that is fixed inside the distal end portion by a fixing member, a non-movable lens frame that is provided in the imaging unit and that is in contact with the fixing member, and a movable lens frame that is provided in the non-movable lens frame and that is slidable along an inner surface of the non-movable lens frame, in which a notch portion is located at point symmetric positions where an imaging optical axis of the imaging unit passes, with respect to a fixed position by the fixing member, and is provided in at least one of portions where the distal end portion and the non-movable lens frame, or the non-movable lens frame and the movable lens frame are in contact with each other. A moving object unit that performs a zooming function or a focusing function is coupled to the movable lens frame.

### SUMMARY OF THE INVENTION

In JP2008-110061A described above, the movable lens frame comprises a movable lens, and the moving object unit is coupled to the movable lens frame. The movable lens frame is movable forward and backward in an imaging optical axis direction in a rear-group lens frame that is the non-movable lens frame. The movable lens frame is provided between a front-group lens and a rear-group lens, and the rear-group lens is held by the rear-group lens frame.

Currently, the optical performance required for an endoscope is increasing. The optical performance is affected by the accuracy of the lens, assembly accuracy, and the like. However, JP2008-110061A does not consider adjusting positions of the front-group lens and the rear-group lens and does not allow for the adjustment of the positions of the front-group lens and the rear-group lens in a case where the required optical performance is not obtained. Therefore, in JP2008-110061A, it is difficult to address a case where the required optical performance is not obtained.

An object of the present invention is to provide an endoscope imaging device and an endoscope capable of adjusting a position of a fixed lens along an optical axis.

In order to achieve the above-described object, according to Invention [1], there is provided an endoscope imaging device that acquires an image of an observation target, comprising: at least one movement group including a movable lens that is movable along an optical axis; a plurality of fixed groups including a fixed lens whose position is fixed with respect to the optical axis; a sensor that receives light which has passed through the movable lens of the movement group and the fixed lens of the fixed group and that images the observation target; a lens drive portion that moves the movement group along the optical axis; and a lens barrel body provided with the movement group, the fixed group, and the lens drive portion, in which at least one of the plurality of fixed groups is an adjustment group, the adjustment group includes a cylindrical holding part inside which the fixed lens is provided, the holding part includes a grip part provided at one end part of the holding part and an end surface provided at the other end part opposite to the one end part in an optical axis direction, and the grip part includes a projecting portion or a recessed portion whose diameter changes with respect to a radial direction of the holding part, and the adjustment group is configured such that the end surface provided at the other end part of the holding part is disposed at a position spaced apart from the lens barrel body or the fixed group in the optical axis direction, and the grip part is disposed at a position spaced apart from the lens barrel body or the fixed group in the optical axis direction and protrudes from the lens barrel body or the fixed group, and a side surface of the holding part is fixed to the lens barrel body or the fixed group.

According to Invention [2], in the endoscope imaging device described in Invention [1], the adjustment group is provided at an end part of the lens barrel body on an observation target side or at an end part of the lens barrel body on a sensor side.

According to Invention [3], in the endoscope imaging device described in Invention [1] or [2], the grip part includes at least one projecting portion or recessed portion, or at least one hole, each of which is provided in a circumferential direction of the holding part, the at least one projecting portion or recessed portion having a diameter that changes with respect to the radial direction of the holding part.

According to Invention [4], in the endoscope imaging device described in any one of Inventions [1] to [3], among the plurality of fixed groups, a fixed group other than the adjustment group is mechanically positioned in the optical axis direction.

According to Invention [5], in the endoscope imaging device described in any one of Inventions [1] to [4], the fixed group is provided at the end part of the lens barrel body on the observation target side, and the adjustment group is provided at the end part of the lens barrel body on the sensor side.

According to Invention [6], there is provided an endoscope comprising: the endoscope imaging device according to any one of Inventions [1] to [5].

According to the present invention, it is possible to provide an endoscope imaging device and an endoscope capable of adjusting the position of the fixed lens along the optical axis. As a result, for example, in a case where the required optical performance is not obtained, the position of the fixed lens can be adjusted along the optical axis.

### BRIEF DESCRIPTION OF THE DRAWINGS

FIG. 1 is a schematic diagram showing an example of an endoscope system of an embodiment of the present invention.

FIG. 2 is a schematic side cross sectional view showing a first example of an endoscope imaging device of the embodiment of the present invention.

FIG. 3 is a schematic side cross sectional view showing a first example of an adjustment group of the endoscope imaging device of the embodiment of the present invention.

FIG. 4 is a schematic plan view showing the first example of the adjustment group of the endoscope imaging device of the embodiment of the present invention.

FIG. 5 is a schematic side cross sectional view showing a second example of the endoscope imaging device of the embodiment of the present invention.

FIG. 6 is an enlarged schematic side cross sectional view showing a first fixed group and an adjustment group of the second example of the endoscope imaging device of the embodiment of the present invention.

FIG. 7 is a schematic partial side cross sectional view showing a second example of a grip part of the adjustment group of the endoscope imaging device of the embodiment of the present invention.

FIG. 8 is a schematic partial side cross sectional view showing a third example of the grip part of the adjustment group of the endoscope imaging device of the embodiment of the present invention.

FIG. 9 is a schematic plan view showing the third example of the grip part of the adjustment group of the endoscope imaging device of the embodiment of the present invention.

FIG. 10 is a schematic partial side cross sectional view showing a fourth example of the grip part of the adjustment group of the endoscope imaging device of the embodiment of the present invention.

FIG. 11 is a schematic partial side cross sectional view showing a fifth example of the grip part of the adjustment group of the endoscope imaging device of the embodiment of the present invention.

FIG. 12 is a schematic plan view showing the fifth example of the grip part of the adjustment group of the endoscope imaging device of the embodiment of the present invention.

FIG. 13 is a schematic plan view showing a sixth example of the grip part of the adjustment group of the endoscope imaging device of the embodiment of the present invention.

### DESCRIPTION OF THE PREFERRED EMBODIMENTS

Hereinafter, based on suitable embodiments shown in the accompanying drawings, an endoscope imaging device and an endoscope of an embodiment of the present invention will be described in detail.

It should be noted that the drawings to be described below are illustrative examples illustrating the present invention and are not intended to limit the present invention to the drawings to be shown below.

The terms “specific angle”, “parallel”, “perpendicular”, “orthogonal”, and the like in the following description include error ranges generally allowed in the relevant technical field.

### Endoscope System

An endoscope system images an observation site such as an inside of a subject's body that is an observation target by irradiating the observation site with illumination light (not shown), generates a display image of the observation site based on an image signal obtained by the imaging, and displays the display image.

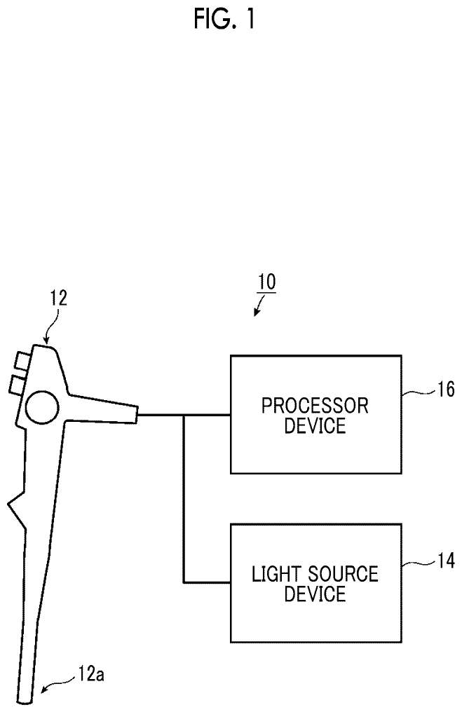

FIG. 1 is a schematic diagram showing an example of the endoscope system of the embodiment of the present invention.

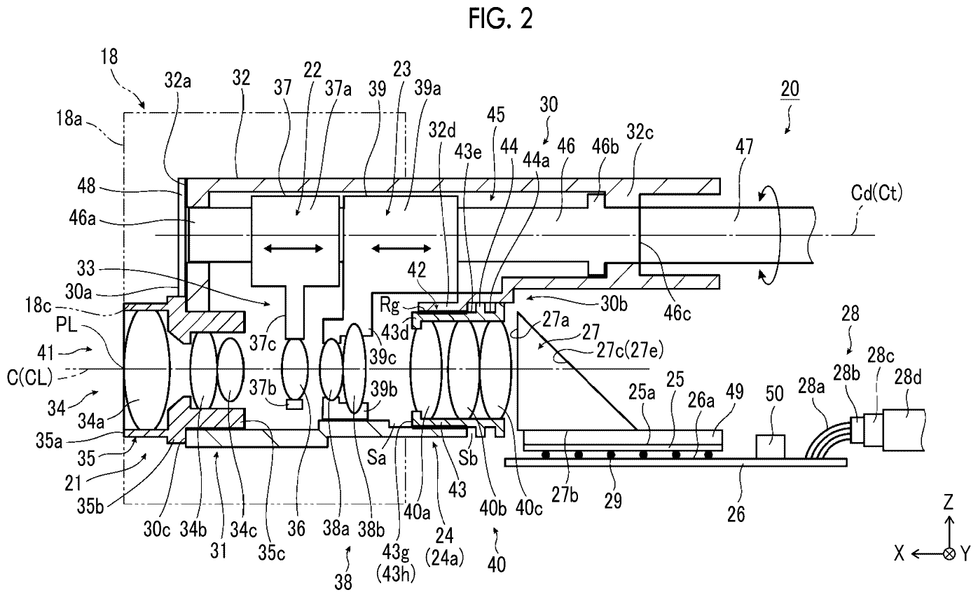

An endoscope system 10 includes an endoscope 12, a light source device 14, and a processor device 16. The endoscope system 10 has a configuration similar to a general endoscope, except for a portion of an endoscope imaging device 20 (refer to FIG. 2) of the endoscope 12, which will be described below.

The endoscope system 10 may further comprise a water supply tank that stores washing water or the like, a suction pump that suctions a suction substance in a body cavity (also including supplied washing water or the like), and the like. Further, a supply pump that supplies washing water inside the water supply tank or a gas such as external air to a pipe line (not shown) inside the endoscope, and the like may be provided.

The endoscope 12 includes, although not shown in detail, an insertion part that is inserted into the subject, an operation part that is contiguous to the insertion part, and a universal cord that extends from the operation part, and the insertion part is composed of a distal end portion, a bending portion that is contiguous to the distal end portion, and a soft portion that connects the bending portion and the operation part to each other.

A distal end portion 12a of the endoscope 12 is provided with an illumination optical system that emits illumination light for illuminating the observation site, a sensor and an imaging optical system that capture a subject image of the observation site, or the like. The bending portion is configured to bend in a direction orthogonal to a longitudinal axis of the insertion part, and a bending motion of the bending portion is operated at the operation part. Additionally, the soft portion is configured to be relatively flexible to the extent that the soft portion can deform to follow the shape of an insertion path of the insertion part.

The operation part is provided with a button for operating an imaging motion of the endoscope imaging device 20 (refer to FIG. 2) of the distal end portion 12a, a knob for operating the bending motion of the bending portion, or the like. In addition, the operation part is provided with an introduction port through which a treatment tool such as an electric scalpel is introduced, and a treatment tool channel that reaches the distal end portion from the introduction port and that allows the treatment tool such as a forceps to be inserted is provided inside the insertion part.

A connector is provided at a terminal of the universal cord, and the endoscope 12 is connected, via the connector, to the light source device 14 that generates illumination light which is emitted from the illumination optical system of the distal end portion and to the processor device 16 that processes a video signal which is acquired by the endoscope imaging device 20 (refer to FIG. 2) of the distal end portion 12a. At least one of signals or power is transmitted between the endoscope imaging device 20 (refer to FIG. 2) and the processor device 16 via an electrical wire group.

The processor device 16 processes the input video signal to generate video data of the observation site, and displays the generated video data on the monitor (not shown) or records the generated video data on a storage medium such as a hard disk. The processor device 16 may be configured with a processor such as a personal computer.

The light source device 14 is used to illuminate an observation target site in the body cavity by generating illumination light, such as white light consisting of three primary color lights such as red light (R), green light (G), and blue light (B), or specific wavelength light, to supply the generated light to the endoscope 12, by propagating the supplied light through a light guide or the like in the endoscope 12, and by emitting the light from the illumination optical system of the distal end portion of the insertion part of the endoscope 12, in order to acquire the image signal of the observation target by imaging the observation target site in the body cavity with the endoscope imaging device 20 (refer to FIG. 2) of the endoscope 12.

The light guide or the electrical wire group (signal cable) is housed inside the insertion part, the operation part, and the universal cord. The illumination light generated by the light source device 14 is guided to the illumination optical system of the distal end portion via the light guide (not shown), and light is emitted from a distal end surface 18a (refer to FIG. 2) of the endoscope imaging device 20 (refer to FIG. 2).

The distal end surface 18a of the endoscope imaging device 20 is provided with, for example, a forceps port (not shown), an observation window 18c (refer to FIG. 2), and the light guide (not shown). In addition to these, the distal end surface 18a may be provided with an air and liquid supply nozzle (not shown).

A forceps pipe (not shown) is connected to the forceps port integrally or separately. A forceps treatment tool, an injection needle, or the like is stored in the forceps pipe. The forceps treatment tool, the injection needle, or the like is configured to protrude to the outside from the forceps port.

The light guide is optically connected to the light source device 14 (refer to FIG. 1), guides illumination light emitted from the light source device 14, and emits the illumination light toward the observation target.

### First Example of Endoscope Imaging Device

FIG. 2 is a schematic side cross sectional view showing a first example of the endoscope imaging device of the embodiment of the present invention.

The endoscope imaging device 20 shown in FIG. 2 acquires an image of the observation target and has an optical zooming function or a focusing function.

The endoscope imaging device 20 includes at least one movement group including a movable lens that is movable along an optical axis C, a plurality of fixed groups including a fixed lens whose position is fixed with respect to the optical axis C, a sensor 25 that receives light which has passed through the movable lens of the movement group and the fixed lens of the fixed group and that images the observation target, a lens drive portion 45 that moves the movement group along the optical axis, and a lens barrel body 30 provided with the movement group, the fixed group, and the lens drive portion.

Specifically, as shown in FIG. 2, the endoscope imaging device 20 includes a first fixed group 21, a first movement group 22, a second movement group 23, and a second fixed group 24, and includes the lens barrel body 30.

A distal end side of the endoscope imaging device 20 is a distal end body 18 side.

The lens barrel body 30 is composed of a lens storage part 31, a coupling part 33, and a housing 32.

The observation window 18c is provided with the tubular lens storage part 31. In a case where the distal end surface 18a of a distal end body 18 is viewed from the optical axis direction, the tubular lens storage part 31 and the tubular housing 32 are arranged in the distal end body 18 such that, for example, a central axis CL of the tubular lens storage part 31 and a central axis Ct of the tubular housing 32 are parallel to each other. The lens storage part 31 and the housing 32 are coupled in a state in which the lens storage part 31 and the housing 32 communicate with each other through the coupling part 33 in which two plate materials are disposed parallel.

In a case where the lens storage part 31, the housing 32, and the coupling part 33 are viewed from a distal end surface 18a side of the endoscope imaging device 20, two circles, which are spaced apart from each other, are disposed between two edges whose centers are parallel to each other and connected by the two edges, and form a shape without any arcs inside the two edges. In other words, the arcs are connected to both sides of the two parallel edges, and the shape is composed of the arcs except for the two edges.

In the lens barrel body 30, an end part on the distal end body 18 side is an end part 30a on an observation target side. The sensor 25 is provided at an end part 30b of the lens barrel body 30 opposite to the end part 30a on the observation target side.

In the example shown in FIG. 2, among the plurality of fixed groups of the first fixed group 21 and the second fixed group 24, the second fixed group 24 is an adjustment group 24a.

The first fixed group 21, the first movement group 22, the second movement group 23, and the second fixed group 24 are provided in the lens barrel body 30 in this order along a direction from the end part 30a of the lens barrel body 30 on a subject side toward the end part 30b on a sensor 25 side.

The first fixed group 21 is provided at the end part 30a of the lens barrel body 30 on the observation target side. The second fixed group 24, that is, the adjustment group 24a, is provided at the end part 30b of the lens barrel body 30 on the sensor 25 side.

The first movement group 22 and the second movement group 23 are provided inside the lens barrel body 30, and the first movement group 22 is disposed on the observation target side of the lens barrel body 30.

The first fixed group 21 includes a first fixed lens 34 and a lens holding member 35 that holds the first fixed lens 34. The first fixed lens 34 includes, for example, three lenses 34a, 34b, and 34c. The three lenses 34a, 34b, and 34c are collectively held in one lens holding member 35 in a state in which the positions thereof are fixed with respect to the optical axis C with the optical axes C thereof aligned.

The above-described lens holding member 35 is fitted into an end surface 30c of the lens storage part 31 of the lens barrel body 30 on the subject side.

The lens holding member 35 includes a cylindrical holding portion 35a, a flange 35b, and a cylindrical storage portion 35c. The flange 35b projects in a radial direction of the first fixed lens 34 with respect to the storage portion 35c. The flange 35b is in contact with the end surface 30c of the lens storage part 31 of the lens barrel body 30, and the lens holding member 35 of the first fixed group 21 is mechanically positioned by the lens barrel body 30 in the optical axis direction. Consequently, the lens holding member 35 keeps the holding portion 35a outside the lens barrel body 30. The storage portion 35c is fitted into the end surface 30c of the lens storage part 31 on the subject side. The lens 34a is held by the holding portion 35a, and the lens 34b and the lens 34c are held by the storage portion 35c.

The fixed group being mechanically positioned in the optical axis direction means that the position of the adjustment group is fixed by being in contact with the lens barrel body 30 in the optical axis direction.

The end surface 30c of the lens storage part 31 on the subject side and an end surface 32a of the housing 32 on the subject side are located on the same plane, but the lens holding member 35 protrudes from the end surface 30c of the lens storage part 31 on the subject side. In the first fixed lens 34, the lens 34a is disposed outside the lens storage part 31.

The lens holding member 35 can be rotated around the optical axis C by the flange 35b and the cylindrical storage portion 35c. Therefore, the first fixed lens 34 can be adjusted in a circumferential direction, and the modulation transfer function (MTF) as optical performance and the lens eccentricity can be adjusted. After the circumferential position of the first fixed lens 34 is adjusted, the flange 35b of the lens holding member 35 and the end surface 30c are fixed to each other by using, for example, an adhesive. An adhesive used to fix the lens barrel body 30 and the adjustment group 24a, which will be described below, can be used as the adhesive.

The MTF and the lens eccentricity can be performed, for example, by imaging a test chart, generating video data of the test chart using the processor device 16 (refer to FIG. 1), and displaying the video data of the test chart on the monitor (not shown). By adjusting the lens eccentricity, so-called unbalanced blurriness can be eliminated.

The first movement group 22 includes a first movable lens 36 and a first movable lens holding part 37, and the first movable lens 36 is movable along the optical axis C.

The first movable lens 36 is, for example, a single lens. The first movable lens holding part 37 includes a tubular guide portion 37a, a lens frame 37b, and a coupling portion 37c that couples the guide portion 37a and the lens frame 37b to each other, and the guide portion 37a, the lens frame 37b, and the coupling portion 37c are integrally formed. In more detail, the above-described first movable lens 36 is held by the lens frame 37b.

The second movement group 23 includes a second movable lens 38 and a second movable lens holding part 39, and the second movable lens 38 is movable along the optical axis C.

The second movable lens 38 includes, for example, two lenses 38a and 38b. The two lenses 38a and 38b of the second movable lens 38 are held by the second movable lens holding part 39. The second movable lens holding part 39 includes a tubular guide portion 39a, a lens frame 39b, and a coupling portion 39c that couples the guide portion 39a and the lens frame 39b to each other, and the guide portion 39a, the lens frame 39b, and the coupling portion 39c are integrally formed. In more detail, the two lenses 38a and 38b of the above-described second movable lens 38 are collectively held in one lens frame 39b with the optical axes C thereof aligned.

The second fixed group 24, that is, the adjustment group 24a, includes a second fixed lens 40 and a fixed lens holding member 42.

The second fixed lens 40 includes, for example, three lenses 40a, 40b, and 40c. The three lenses 40a, 40b, and 40c are collectively held in one fixed lens holding member 42 in a state in which the positions thereof are fixed with respect to the optical axis C with the optical axes C thereof aligned.

In the first fixed lens 34, the first movable lens 36, the second movable lens 38, and the second fixed lens 40, the optical axes C of the respective lenses are aligned with each other. The first fixed lens 34, the first movable lens 36, the second movable lens 38, and the second fixed lens 40 are disposed in the lens storage part 31 with the optical axes C thereof aligned with the central axis CL of the lens storage part 31.

An imaging lens 41 is composed of the first fixed lens 34, the first movable lens 36, the second movable lens 38, and the second fixed lens 40. The first fixed lens 34, the first movable lens 36, the second movable lens 38, and the second fixed lens 40 are optical elements that form an image of incident light on a light-receiving surface 25a of the sensor 25. The first fixed lens 34, the first movable lens 36, the second movable lens 38, and the second fixed lens 40 form the subject image on the light-receiving surface 25a of the sensor 25.

The first fixed lens 34, the first movable lens 36, the second movable lens 38, and the second fixed lens 40 are held such that the optical axes C are perpendicular to an incidence surface 27a of a prism 27, which will be described below. It should be noted that the configuration of the imaging lens 41 is not particularly limited to the above-described configuration. In addition, each lens constituting the imaging lens 41 may be a convex lens or a concave lens.

Here, a direction parallel to the optical axes C of the first fixed lens 34, the first movable lens 36, the second movable lens 38, and the second fixed lens 40 is denoted as an X direction. Among two directions orthogonal to the optical axis C, one is denoted a Y direction, and the remaining one is denoted as a Z direction. The above-described X direction corresponds to the optical axis direction. An axial direction such as the optical axis direction refers to an axis extension direction. The optical axis direction is an extension direction of the optical axis C.

In addition, the endoscope imaging device 20 includes the lens drive portion 45 that moves the first movement group 22 and the second movement group 23 along the optical axis C in order to exhibit an optical zooming function or a focusing function. Being movable along the optical axis C refers to being movable in the optical axis direction.

The lens drive portion 45 is stored in the housing 32. The lens drive portion 45 includes a camshaft 46. For example, the camshaft 46 has a columnar shape and is disposed with a rotation axis Cd thereof aligned with the central axis Ct of the housing 32. Consequently, the central axis CL of the lens storage part 31 and the central axis Ct of the housing 32 are parallel to the optical axis C.

A drive member 47 is connected to an end part 46c of the camshaft 46 opposite to the above-described end surface 32a. The camshaft 46 rotates about the rotation axis Cd by the rotation of the drive member 47.

The drive member 47 is connected to, for example, a drive unit such as a motor. The drive member 47 is rotated by the motor. In addition, for example, the drive member 47 is made of a flexible member. In more detail, the drive member 47 is made of a wire.

A distal end support portion 48 is provided on the end surface 32a of the housing 32 on the subject side. The camshaft 46 is rotatably supported by the distal end support portion 48.

The camshaft 46 includes a flange 46b that is provided in the vicinity of the end part 46c and that protrudes outward along an outer periphery. A locking ring 32c opposite to the above-described end surface 32a is provided inside the housing 32 in which the lens drive portion 45 is stored.

The flange 46b of the camshaft 46 is disposed on an end surface 32a side of the housing 32 with respect to the locking ring 32c and is stored inside the housing 32. The friction between the flange 46b of the camshaft 46 and the locking ring 32c is small and does not hinder the rotation of the camshaft 46. In addition, the locking ring 32c prevents the camshaft 46 from coming off.

On the camshaft 46, the first movable lens holding part 37 and the second movable lens holding part 39 are provided between a distal end 46a and the flange 46b in a rotation axis direction. The first movable lens holding part 37 and the second movable lens holding part 39 are disposed across the lens storage part 31, the coupling part 33, and the housing 32.

For example, a first cam groove (not shown) for moving the first movable lens 36 along the optical axis direction and a second cam groove (not shown) for moving the second movable lens 38 along the optical axis direction are provided on an outer peripheral surface of the camshaft 46 between the distal end 46a and the flange 46b described above. The rotation axis direction is a direction parallel to the optical axis direction.

The camshaft 46 is slidably inserted through the guide portion 37a of the first movable lens holding part 37. The guide portion 37a of the first movable lens holding part 37 is provided with a cam pin (not shown) that is engaged with the first cam groove (not shown).

In addition, the camshaft 46 is slidably inserted through the guide portion 39a of the second movable lens holding part 39. The guide portion 39a of the second movable lens holding part 39 is provided with a cam pin (not shown) that is engaged with the second cam groove (not shown).

In the first movement group 22, in a case where the camshaft 46 rotates in a state in which the cam pin (not shown) of the first movable lens holding part 37 is engaged with the first cam groove (not shown), the first movable lens holding part 37 moves along the rotation axis direction (optical axis direction) of the camshaft 46 according to the direction of the rotation. Further, the first movable lens holding part 37 can be stopped and positioned at a predetermined position in the rotation axis direction (optical axis direction) of the camshaft 46. In this case, the coupling portion 37c of the first movable lens holding part 37 passes through the coupling part 33, and the first movable lens 36 moves along the rotation axis direction (optical axis direction) of the camshaft 46 and is positioned at a predetermined position.

In the second movement group 23, in a case where the camshaft 46 rotates in a state in which the cam pin (not shown) of the second movable lens holding part 39 is engaged with the second cam groove (not shown), the second movable lens holding part 39 moves along the rotation axis direction (the optical axis direction) of the camshaft 46 according to the direction of the rotation. Further, the second movable lens holding part 39 can be stopped and positioned at a predetermined position in the rotation axis direction (optical axis direction) of the camshaft 46. In this case, the coupling portion 39c of the second movable lens holding part 39 passes through the coupling part 33, and the second movable lens 38 moves along the rotation axis direction (optical axis direction) of the camshaft 46 and is positioned at a predetermined position.

As described above, since the first movement group 22 can move the first movable lens 36 along the optical axis direction, and the second movement group 23 can move the second movable lens 38 along the optical axis direction, it is possible to change the focal length of the imaging lens 41, thereby enabling zooming imaging and focusing.

The endoscope imaging device 20 includes, for example, a circuit board 26, the prism 27, and a signal cable 28, in addition to the sensor 25 described above.

The sensor 25 receives the light that has passed through the first movable lens 36 of the first movement group 22, the second movable lens 38 of the second movement group 23, the first fixed lens 34 of the first fixed group 21, and the second fixed lens 40 of the second fixed group 24, and images the observation target.

The sensor 25 is disposed at the end part 30b of the lens barrel body 30 on a side opposite to the subject side as described above.

The light that has passed through the first movable lens 36 of the first movement group 22, the second movable lens 38 of the second movement group 23, the first fixed lens 34 of the first fixed group 21, and the second fixed lens 40 of the second fixed group 24 is also referred to as light that has passed through the imaging lens 41.

The sensor 25 includes the light-receiving surface 25a that receives the above-described light that has passed the imaging lens 41. The above-described light that has passed through the imaging lens 41 is, for example, light that has formed an image of the observation target, and is imaging light of the observation target.

The sensor 25 captures a subject image by converting light received on the light-receiving surface 25a into an electrical signal through photoelectric conversion, for example. In more detail, in the sensor 25, the above-described imaging light of the observation target is incident on the light-receiving surface 25a, and the imaging light is photoelectrically converted, thereby capturing the subject image. The sensor 25 is, for example, a conventionally known photoelectric conversion element, and a charge coupled device (CCD)-type image sensor or a complementary metal oxide semiconductor (CMOS) image sensor can be used.

As shown in FIG. 2, the sensor 25 is electrically connected to a surface 26a of the circuit board 26, for example, via a bump 29 having conductivity. In addition, for example, the sensor 25 is mounted on the circuit board 26 such that the light-receiving surface 25a is parallel to the optical axes C of the first fixed lens 34, the first movable lens 36, the second movable lens 38, and the second fixed lens 40. The mounting refers to being electrically connected.

An underfill layer (not shown) can also be provided between the sensor 25 and the circuit board 26 in order to firmly connect the sensor 25 and the circuit board 26 to each other.

The bump 29 is made of a metal or an alloy. In more detail, the bump 29 is made of solder. The bump 29 formed with solder is also referred to as a solder ball. It should be noted that the bump 29 is not limited to the solder or the like as long as the sensor 25 and the circuit board 26 can be electrically connected to each other. Additionally, the sensor 25 and the circuit board 26 may be electrically connected directly to each other.

Further, the underfill layer mitigates a stress that occurs at a junction portion between the sensor 25 and the circuit board 26, for example, at the bump 29, due to the difference in thermal expansion coefficients between the sensor 25 and the circuit board 26. With the underfill layer, the sensor 25 and the circuit board 26 are firmly connected to each other, and the reliability of the electrical connection is increased, thereby obtaining a high reliable endoscope imaging device 20.

An underfill agent constituting the underfill layer is not particularly limited, and an underfill agent that can be used as a sealing resin between the sensor 25 and the circuit board 26 can be appropriately used. For example, a one-pack heat-curable epoxy resin is used as the underfill agent. In this case, the underfill agent is supplied and then is heated and held at a predetermined temperature, thereby forming the underfill layer.

The circuit board 26 is a substrate on which the sensor 25 is mounted. Additionally, in the circuit board 26, for example, an electronic component 50 is mounted on the surface 26a of the circuit board 26, in addition to the sensor 25. The electronic component 50 is a component for driving the sensor 25 and is not particularly limited, and examples thereof include a voltage regulator, a resistor, and a capacitor. The voltage regulator is a device that stabilizes a voltage to the sensor 25, and outputs a constant voltage to the sensor 25.

The circuit board 26 shown in FIG. 2 has a flat plate shape, but the present invention is not limited to this.

The circuit board 26 is composed of, for example, a printed wiring board, but may also be composed of a flexible substrate. In this case, the circuit board 26 is composed of, for example, a flexible printed board.

In addition, a plurality of connection terminals (not shown) through which signals or power is input and output with respect to the sensor 25 and the electronic component 50 are provided on the surface 26a of the circuit board 26. A signal line 28a of the signal cable 28 is electrically connected to the connection terminal. Consequently, the sensor 25 and the signal cable 28 are electrically connected to each other. The light is converted into the electrical signal by the sensor 25, and this electrical signal is transmitted via the signal cable 28. The signal cable 28 is inserted through the insertion part, the operation part, the universal cord, and the like of the endoscope and is electrically connected to the processor device 16 (refer to FIG. 1).

The configuration of the signal cable 28 is not particularly limited. For example, as shown in FIG. 2, the signal cable 28 includes a plurality of the signal lines 28a, a covering layer 28b that covers each signal line 28a, a shield conductor 28c provided around the entire periphery of the plurality of signal lines 28a covered with the covering layer 28b, and an outer sheath 28d that covers the shield conductor 28c. The signal cable 28 is a multicore cable including the plurality of signal lines 28a bundled together, provided to be surrounded by the shield conductor 28c, and stored inside the cylindrical outer sheath 28d.

The outer sheath 28d constitutes an outer periphery of the signal cable 28. The covering layer 28b, the shield conductor 28c, and the outer sheath 28d are, for example, cylindrical in shape. In addition, the shield conductor 28c of the signal cable 28 is referred to as a shield. The signal cable 28 includes, for example, five signal lines 28a. The number of signal lines 28a depends on the configuration of the endoscope imaging device 20 and is not particularly limited, and may be two, three, four, or six or more.

The prism 27 guides the light that has passed through the imaging lens 41 to the light-receiving surface 25a of the sensor 25. The prism 27 is, for example, a right-angle prism in which the incidence surface 27a and an emission surface 27b are perpendicular to each other. Additionally, the prism 27 includes an inclined surface 27c connecting the incidence surface 27a and the emission surface 27b to each other. The inclined surface 27c is a reflecting surface 27e of the prism 27.

The prism 27 is disposed at the end part 30b of the lens storage part 31 on the side opposite to the subject side. The prism 27 is disposed such that the incidence surface 27a faces the lens 40c of the second fixed lens 40. Additionally, the prism 27 is disposed such that the emission surface 27b faces the light-receiving surface 25a of the sensor 25.

Moreover, for example, a cover glass 49 is disposed between the emission surface 27b of the prism 27 and the light-receiving surface 25a of the sensor 25. The cover glass 49 protects the light-receiving surface 25a of the sensor 25. The prism 27 and the cover glass 49 are, for example, bonded to each other with a photocurable adhesive. It should be noted that a configuration may also be employed in which the cover glass 49 is not provided.

The prism 27 bends the light that has passed through the imaging lens 41 on the inclined surface 27c, that is, on the reflecting surface 27e, by, for example, 90° to change an optical path, and guides the light to the light-receiving surface 25a of the sensor 25. The light that has been transmitted through the imaging lens 41 is incident on the prism 27 and is reflected on the inclined surface 27c of the prism 27, that is, transmitted light that has been transmitted through the imaging lens 41 is reflected on the reflecting surface 27e, and is incident on the light-receiving surface 25a of the sensor 25. The light that has been transmitted through the imaging lens 41 is light including information on the observation target.

In the endoscope imaging device 20, an observed image captured from the imaging lens 41 to the sensor 25 is formed on the light-receiving surface 25a of the sensor 25 and is converted into the electrical signal, and this electrical signal is output to the processor device 16 (refer to FIG. 1) via the signal cable 28 and is converted into the video signal, thereby displaying an observation image on the monitor connected to the processor device 16.

### Adjustment Group

Hereinafter, the adjustment group 24a will be described.

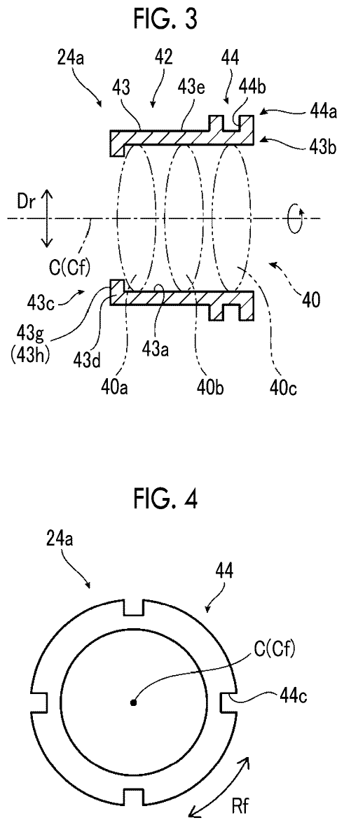

FIG. 3 is a schematic side cross sectional view showing a first example of the adjustment group of the endoscope imaging device of the embodiment of the present invention, and FIG. 4 is a schematic plan view showing the first example of the adjustment group of the endoscope imaging device of the embodiment of the present invention. A grip part 44 shown in FIG. 3 is a first example of a grip part. FIG. 4 is a view of FIG. 3 as viewed from a grip part 44 side. In FIGS. 3 and 4, the same components as those of the endoscope imaging device 20 shown in FIG. 2 are designated by the same reference numerals, and detailed descriptions thereof will be omitted.

The adjustment group 24a shown in FIG. 3 includes the fixed lens holding member 42. The fixed lens holding member 42 comprises a cylindrical holding part 43 in which the second fixed lens 40 is provided in an interior 43a. The holding part 43 comprises the grip part 44 provided at one end part 43b of the holding part 43 and an end surface 43g provided at the other end part 43c opposite to the one end part 43b in the optical axis direction. The grip part 44 comprises a projecting portion or a recessed portion whose diameter changes with respect to a radial direction Dr of the holding part 43.

The grip part 44 is, for example, a flange portion 44a that protrudes in the radial direction Dr of the second fixed lens 40 with respect to the holding part 43. The flange portion 44a is provided to surround the periphery of the holding part 43. In addition, a recessed portion 44b that surrounds the periphery of the holding part 43 is provided in the flange portion 44a.

The flange portion 44a protrudes from a side surface 43e of the holding part 43 and has a larger diameter with respect to the radial direction Dr of the holding part 43. The flange portion 44a corresponds to the projecting portion.

The recessed portion 44b has a smaller diameter with respect to the radial direction Dr of the holding part 43. The recessed portion 44b is parallel to the central axis CL of the holding part 43 as shown in FIG. 3, and has, for example, a quadrangular cross sectional shape in a cross section including the central axis CL and includes a flat bottom surface on an interior 43a side. The radial direction Dr of the holding part 43 is the radial direction Dr of the second fixed lens 40.

The grip part 44 is used in a case where the adjustment group 24a is moved in the optical axis direction. For example, a jig (not shown) is hooked on the flange portion 44a or the recessed portion 44b to move the adjustment group 24a in the optical axis direction. Consequently, for example, in a case where the required optical performance is not obtained, the position of the fixed lens can be adjusted along the optical axis C, and the angle of view can be adjusted as the optical performance. After the position of the fixed lens is adjusted along the optical axis C, the adjustment group 24a is fixed to the lens barrel body 30 by using, for example, an adhesive. The angle of view can be adjusted, for example, in a state in which the video data generated by the processor device 16 (refer to FIG. 1) is displayed on the monitor (not shown).

By designating at least one group from among the fixed groups as the adjustment group 24a in this way, the position of the fixed lens can be adjusted along the optical axis C. As a result, for example, in a case where the required optical performance is not obtained, the position of the fixed lens can be adjusted along the optical axis C.

In addition, since the holding part 43 has a cylindrical shape, the adjustment group 24a can also be rotated around a central axis Cf with the central axis Cf as the rotation axis. Consequently, the second fixed lens 40 can be adjusted in the circumferential direction, and the MTF as the optical performance and the lens eccentricity can be adjusted. After the circumferential position of the second fixed lens 40 is adjusted, the adjustment group 24a is fixed to the lens barrel body 30 by using, for example, an adhesive.

It is preferable that the flange portion 44a comprises at least one projecting portion or recessed portion, or at least one hole, each of which is provided in a circumferential direction Rf of the central axis Cf of the holding part 43, and the at least one projecting portion or recessed portion has a diameter that changes with respect to the radial direction Dr of the holding part 43. The circumferential direction Rf of the central axis Cf of the holding part 43 is also simply referred to as the circumferential direction Rf of the holding part 43.

In more detail, as shown in FIG. 4, it is preferable that four recessed portions 44c are provided at equal intervals in the circumferential direction Rf of the holding part 43. That is, it is preferable that the recessed portions 44c are provided at 90° intervals around the central axis Cf. The recessed portion 44c has a smaller diameter with respect to the radial direction Dr of the holding part 43.

The recessed portion 44c has, for example, a quadrangular opening shape and includes a flat bottom surface on the interior 43a side. The circumferential direction Rf of the central axis Cf of the holding part 43 is also the circumferential direction Rf of the second fixed lens 40.

The jig (not shown) can be hooked on the recessed portions 44c to easily rotate the adjustment group 24a around the central axis Cf. As a result, the MTF as the optical performance and the lens eccentricity can be easily adjusted.

Additionally, at the other end part 43c, a ring member 43d is provided at the opening of the holding part 43. The ring member 43d blocks a part of the opening of the holding part 43. The ring member 43d functions as a stopper for retaining the lens 40a in the interior 43a. In the first example of the endoscope imaging device, an end surface 43h of the ring member 43d opposite to the one end part 43b in the optical axis direction is the above-described end surface 43g provided at the other end part 43c of the holding part 43.

As shown in FIG. 2, the adjustment group 24a is disposed at a position where the end surface 43g provided at the other end part 43c of the holding part 43 is spaced apart from the lens barrel body 30 in the optical axis direction. As described above, the end surface 43g provided at the other end part 43c of the holding part 43 is the end surface 43h of the ring member 43d and is a surface different from the side surface 43e. The end surface 43g (the end surface 43h of the ring member 43d of the holding part 43) provided at the other end part 43c of the holding part 43 is not in contact with anywhere on the lens barrel body 30 in the optical axis direction. The end surface 43g (the end surface 43h of the ring member 43d of the holding part 43) provided at the other end part 43c of the holding part 43 of the adjustment group 24a has a gap Sa with the lens barrel body 30 in the optical axis direction.

The grip part 44 is disposed at a position spaced apart from the lens barrel body 30 in the optical axis direction and protrudes from the lens barrel body 30. The flange portion 44a that is the grip part 44 of the adjustment group 24a is not in contact with anywhere on the lens barrel body 30 in the optical axis direction. The grip part 44 has a gap Sb with the lens barrel body 30 in the optical axis direction. In such a state, the side surface 43e of the holding part 43 is fixed to the lens barrel body 30.

A portion where a support portion 32d of the lens barrel body 30 and the side surface 43e of the holding part 43 of the adjustment group 24a are in contact with each other is a fitting portion Rg.

In addition, in a case where there is a portion protruding like the flange portion 44a in the adjustment group 24a, in addition to the ring member 43d of the adjustment group 24a described above, the flange portion 44a that is the grip part 44 of the adjustment group 24a is not in contact with anywhere on the lens barrel body 30 in the optical axis direction.

As described above, in the adjustment group 24a, a state in which the end surface 43g provided at the other end part 43c of the holding part 43 is disposed at a position spaced apart from the lens barrel body 30 in the optical axis direction, and the grip part 44 is disposed at a position spaced apart from the lens barrel body 30 in the optical axis direction and protrudes from the lens barrel body 30 is simply referred to as a set position of the holding part 43.

The lens barrel body 30 and the adjustment group 24a are fixed to each other by using, for example, an adhesive. In a case of being bonded and fixed to each other, the adhesive is in a cured state. The adhesive is not particularly limited, and for example, a photocurable adhesive or an instant adhesive is used. Consequently, the lens barrel body 30 and the adjustment group 24a can be reliably fixed to each other in a short time in a state in which a positional relationship between the lens barrel body 30 and the adjustment group 24a is maintained. This makes manufacturing easier, and fewer jigs are needed to fix the lens barrel body 30 and the adjustment group 24a.

The photocurable adhesive is, for example, an adhesive that is cured by ultraviolet light with a wavelength of about 100 to 400 nm, visible light with a wavelength of about more than 400 and less than 780 nm, infrared light with a wavelength of about 780 nm to 1 mm, or the like.

The photocurable adhesive is, for example, an epoxy resin-based photocurable adhesive, an acrylic resin-based photocurable adhesive, or a silicone-based photocurable adhesive.

In the endoscope imaging device 20 shown in FIG. 2, the second fixed group 24 is designated as the adjustment group 24a from among the first fixed group 21 and the second fixed group 24, but the present invention is not limited to this, and the first fixed group 21 may be designated as the adjustment group instead of the second fixed group 24.

In addition, both the first fixed group 21 and the second fixed group 24 may be designated as the adjustment groups. By designating both the first fixed group 21 and the second fixed group 24 as the adjustment groups, even more adjustments are possible.

### Second Example of Endoscope Imaging Device

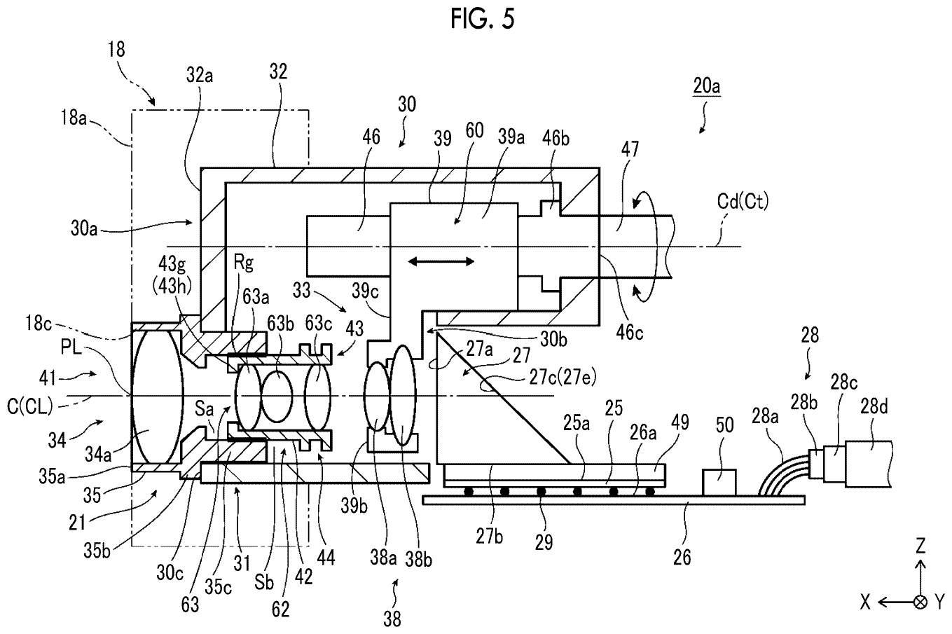

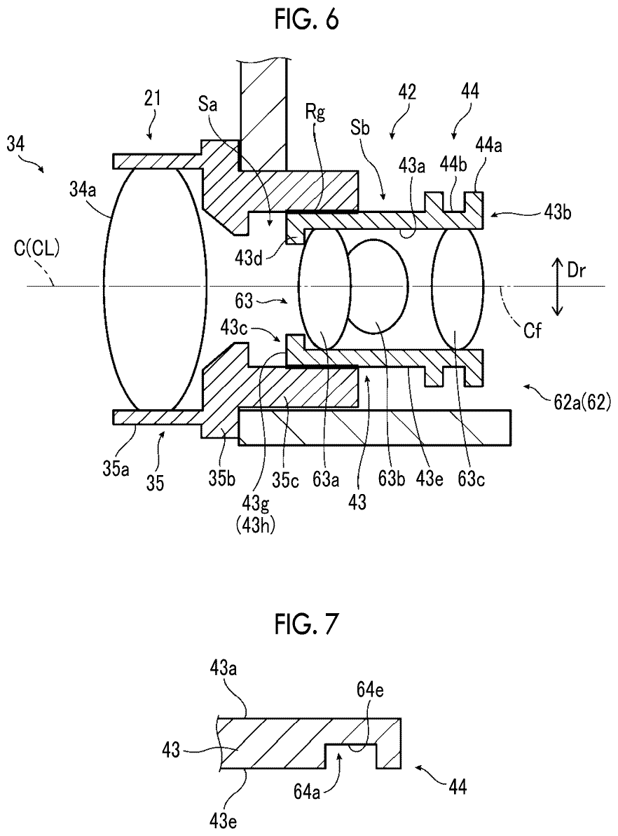

FIG. 5 is a schematic side cross sectional view showing a second example of the endoscope imaging device of the embodiment of the present invention, and FIG. 6 is an enlarged schematic side cross sectional view showing a first fixed group and an adjustment group of the second example of the endoscope imaging device of the embodiment of the present invention.

In FIGS. 5 and 6, the same components as those of the endoscope imaging device 20 shown in FIG. 2 are designated by the same reference numerals, and detailed descriptions thereof will be omitted.

An endoscope imaging device 20a shown in FIG. 5 is different from the endoscope imaging device 20 shown in FIG. 2 in that one movement group 60 is provided, an adjustment group 62a is fixed to the first fixed group 21, and the first fixed lens 34 includes one lens 34a, and the other configurations are the same as those of the endoscope imaging device 20 shown in FIG. 2.

In the endoscope imaging device 20a, the first fixed group 21, the adjustment group 62a, and the movement group 60 are provided in this order along a direction from the end part 30a of the lens barrel body 30 on the subject side toward the end part 30b on the sensor 25 side. The first fixed group 21 is provided at the end part 30a of the lens barrel body 30 on the observation target side. The adjustment group 62a and the movement group 60 are provided inside the lens barrel body 30. The adjustment group 62a is a second fixed group 62.

The first fixed group 21 includes the first fixed lens 34 and the lens holding member 35 that holds the first fixed lens 34. The first fixed lens 34 is composed of, for example, one lens 34a.

The lens holding member 35 includes the cylindrical holding portion 35a, the flange 35b, and the cylindrical storage portion 35c as described above. The storage portion 35c is fitted into the end surface 30c of the lens storage part 31 on the subject side. The lens 34a is held by the holding portion 35a, and the adjustment group 62a is stored in the storage portion 35c.

The movement group 60 is provided on the sensor 25 side with respect to the adjustment group 62a. The adjustment group 62a will be described in detail below.

The movement group 60 has the same configuration as that of the second movement group 23 of the endoscope imaging device 20 shown in FIG. 2.

The adjustment group 62a includes a second fixed lens 63 and the fixed lens holding member 42, similarly to the adjustment group 24a of the endoscope imaging device 20 shown in FIG. 2. The second fixed lens 63 includes, for example, three lenses 63a, 63b, and 63c. The three lenses 63a, 63b, and 63c are collectively held in one fixed lens holding member 42 in a state in which the positions thereof are fixed with respect to the optical axis C with the optical axes C thereof aligned.

For example, among the three lenses 63a, 63b, and 63c, the lenses 63a and 63b are the lenses 34a and 34b of the first fixed lens 34 described above.

In the endoscope imaging device 20a, the imaging lens 41 is composed of the first fixed lens 34, the second fixed lens 63, and the second movable lens 38.

As shown in FIGS. 5 and 6, the adjustment group 62a is disposed at a position where the end surface of the holding part 43 opposite to the sensor 25 (refer to FIG. 2) is spaced apart from the lens holding member 35 in the optical axis direction. In the second example of the endoscope imaging device as well, the end surface 43g provided at the other end part 43c (refer to FIG. 6) of the holding part 43 is the end surface 43h of the ring member 43d, as in the first example of the endoscope imaging device.

The end surface 43g (the end surface 43h of the ring member 43d) provided at the other end part 43c of the holding part 43 is not in contact with anywhere on the lens holding member 35 in the optical axis direction. The end surface 43g (the end surface 43h of the ring member 43d of the adjustment group 62a) provided at the other end part 43c of the holding part 43 has the gap Sa with the lens holding member 35 in the optical axis direction.

The grip part 44 is disposed at a position spaced apart from the lens holding member 35 in the optical axis direction and protrudes from the lens holding member 35. In the endoscope imaging device 20a, the flange portion 44a as the grip part 44 protrudes from the storage portion 35c of the lens holding member 35 toward the sensor 25 side.

The flange portion 44a that is the grip part 44 of the adjustment group 62a is located on the sensor 25 side with respect to the storage portion 35c and is not in contact with anywhere on the lens holding member 35 in the optical axis direction. The grip part 44 has the gap Sb with the lens barrel body 30 in the optical axis direction. In such a state, the side surface 43e of the holding part 43 is fixed to the lens holding member 35. A portion where the storage portion 35c of the lens holding member 35 and the side surface 43e of the holding part 43 of the adjustment group 62a are in contact with each other is the fitting portion Rg.

In addition, as described above, in a case where there is a portion protruding like the flange portion 44a in the adjustment group 62a, in addition to the ring member 43d of the adjustment group 62a described above, the flange portion 44a that is the grip part 44 of the adjustment group 62a is not in contact with anywhere on the first fixed group 21 in the optical axis direction.

In the endoscope imaging device 20a as well, in the adjustment group 62a, for example, a jig (not shown) can be hooked on the recessed portion 44b to move the adjustment group 62a in the optical axis direction, in the same manner as the adjustment group 24a of the endoscope imaging device 20 shown in FIG. 2. Consequently, for example, in a case where the required optical performance is not obtained, the position of the fixed lens can be adjusted along the optical axis C, and the angle of view can be adjusted as the optical performance. After the position of the fixed lens is adjusted along the optical axis C, the adjustment group 62a is fixed to the lens holding member 35 of the first fixed group 21 by using, for example, an adhesive.

In addition, since the holding part 43 has a cylindrical shape, the adjustment group 62a can also be rotated around the central axis Cf with the central axis Cf as the rotation axis. Consequently, the second fixed lens 63 can be adjusted in the circumferential direction, and the MTF as the optical performance and the lens eccentricity can be adjusted. After the circumferential position of the second fixed lens 63 is adjusted, the adjustment group 62a is fixed to the lens holding member 35 of the first fixed group 21 by using, for example, an adhesive.

Further, the jig is hooked on the recessed portion 44c (refer to FIG. 4) of the flange portion 44a that is the grip part 44, so that the adjustment group 62a can be easily rotated around the central axis Cf.

As the adhesive used to bond the adjustment group 62a and the lens holding member 35 of the first fixed group 21, the adhesive used to fix the lens barrel body 30 and the adjustment group 24a described above can be used.

In the endoscope imaging device 20a shown in FIGS. 5 and 6, the second fixed group 62 is designated as the adjustment group 62a from among the first fixed group 21 and the second fixed group 62, but the present invention is not limited to this, and the first fixed group 21 may be designated as the adjustment group.

In addition, both the first fixed group 21 and the second fixed group 62 may be designated as the adjustment groups. By designating both the first fixed group 21 and the second fixed group 62 as the adjustment groups, even more adjustments are possible.

### Other Examples of Adjustment Group

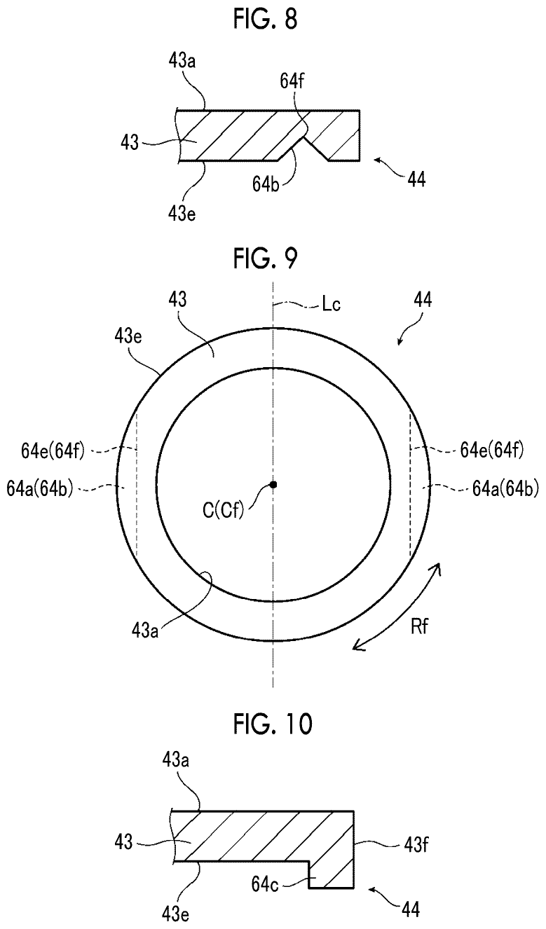

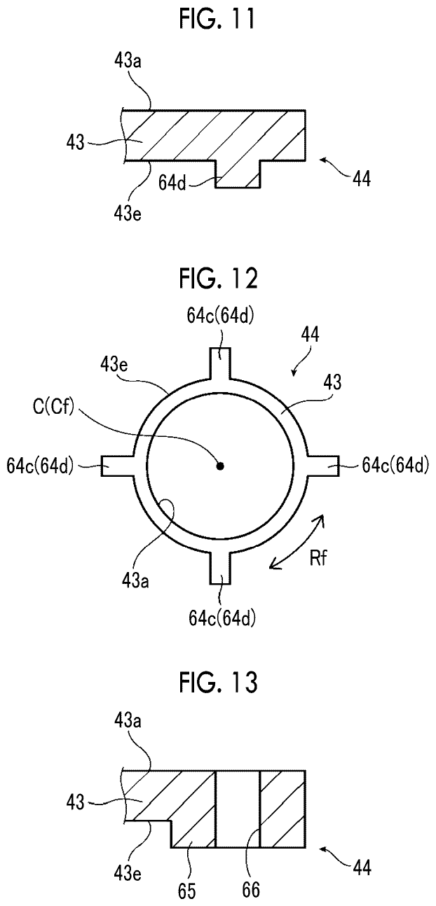

FIG. 7 is a schematic partial side cross sectional view showing a second example of the grip part of the adjustment group of the endoscope imaging device of the embodiment of the invention. FIG. 8 is a schematic partial side cross sectional view showing a third example of the grip part of the adjustment group of the endoscope imaging device of the embodiment of the present invention. FIG. 9 is a schematic plan view showing the third example of the grip part of the adjustment group of the endoscope imaging device of the embodiment of the present invention. FIG. 10 is a schematic partial side cross sectional view showing a fourth example of the grip part of the adjustment group of the endoscope imaging device of the embodiment of the present invention, and FIG. 11 is a schematic partial side cross sectional view showing a fifth example of the grip part of the adjustment group of the endoscope imaging device of the embodiment of the present invention. FIG. 12 is a schematic plan view showing the fifth example of the grip part of the adjustment group of the endoscope imaging device of the embodiment of the present invention. FIG. 13 is a schematic plan view showing a sixth example of the grip part of the adjustment group of the endoscope imaging device of the embodiment of the present invention.

In FIGS. 7 to 13, the same components as those of the adjustment group 24a shown in FIGS. 3 and 4 are designated by the same reference numerals, and the detailed descriptions thereof will be omitted.

The configuration of the grip part 44 of the adjustment group is not limited to the configuration in which the flange portion 44a and the recessed portion 44b described above are provided. For example, the grip part 44 may have a configuration in which a recessed portion 64a is provided as shown in FIG. 7. The recessed portion 64a is disposed, for example, on the side surface 43e of the holding part 43 to surround the periphery of the holding part 43. The recessed portion 64a is parallel to the central axis CL (refer to FIG. 3) of the holding part 43 (refer to FIG. 3), and has, for example, a quadrangular cross sectional shape in the cross section including the central axis CL and includes a flat bottom surface 64e on the interior 43a side.

As shown in FIG. 8, the grip part 44 may have a configuration in which a recessed portion 64b is provided. The recessed portion 64b is disposed, for example, on the side surface 43e of the holding part 43 to surround the periphery of the holding part 43. The recessed portion 64b is parallel to the central axis CL (refer to FIG. 3) of the holding part 43 (refer to FIG. 3), and has, for example, a triangular cross sectional shape in the cross section including the central axis CL, with a vertex directed toward the interior 43a side. A bottom portion 64f on the interior 43a side corresponds to the vertex of the cross sectional shape of the recessed portion 64b.

It should be noted that the cross sectional shape of the recessed portion is not limited to the above-described quadrangle and triangle, and the bottom surface on the interior 43a side may be a curved surface.

In a case where the grip part 44 includes the recessed portions 64a and 64b as shown in FIGS. 7 and 8, for example, as shown in FIG. 9, two recessed portions 64a and 64b are provided symmetrically with respect to a straight line Lc passing through the central axis Cf of the holding part 43. The recessed portions 64a and 64b extend in, for example, a direction parallel to an extension direction of the straight line Lc. The recessed portions 64a and 64b are not provided in the entire region of the holding part 43 in the circumferential direction Rf, but are partially provided in the holding part 43. The jig can be hooked on the recessed portions 64a and 64b to easily move the adjustment group in the optical axis direction. Moreover, the holding part 43 can be easily rotated around the central axis Cf.

The configurations of the recessed portions 64a and 64b are not particularly limited to the configurations shown in FIG. 9. For example, a configuration may also be employed in which the four recessed portions 64a and 64b are provided at equal intervals in the circumferential direction Rf of the holding part 43, that is, at 90° intervals around the central axis Cf of the holding part 43.

As shown in FIGS. 7 and 8, in a case where the adjustment group is at the above-described set position of the holding part 43 in the configuration in which the grip part 44 includes the recessed portions 64a and 64b, the recessed portions 64a and 64b that are the grip part 44 are disposed at positions spaced apart from the lens barrel body or the fixed group in the optical axis direction, protrude from the lens barrel body or the fixed group, and are not in contact with anywhere on the lens barrel body and the fixed group.

In addition, as shown in FIG. 10, the grip part 44 may have a configuration in which a flange portion 64c is provided. The flange portion 64c is disposed at an end part 43f of the holding part 43 opposite to the ring member 43d on the side surface 43e of the holding part 43 to surround the periphery of the holding part 43.

The flange portion 64c is parallel to the central axis CL (refer to FIG. 3) of the holding part 43 (refer to FIG. 3), and has, for example, a quadrangular cross sectional shape in the cross section including the central axis CL, with a surface opposite to the interior 43a being flat.

As shown in FIG. 11, the grip part 44 may have a configuration in which a flange portion 64d is provided. The flange portion 64d is disposed on the side surface 43e of the holding part 43 to surround the periphery of the holding part 43. The flange portion 64d is parallel to the central axis CL (refer to FIG. 3) of the holding part 43 (refer to FIG. 3), and has, for example, a quadrangular cross sectional shape in the cross section including the central axis CL, with a surface opposite to the interior 43a being a flat.

The flange portions 64c and 64d shown in FIGS. 10 and 11 each protrude from the side surface 43e of the holding part 43 and have a larger diameter with respect to the radial direction Dr of the holding part 43. The flange portions 64c and 64d correspond to the projecting portions.

As shown in FIGS. 10 and 11, the configuration in which the flange portions 64c and 64d are provided is, for example, a configuration in which four flange portions 64c and 64d are provided at equal intervals in the circumferential direction Rf of the holding part 43 as shown in FIG. 12. That is, the flange portions 64c and 64d are provided at 90° intervals around the central axis Cf of the holding part 43. The jig can be hooked on the flange portions 64c and 64d to easily move the adjustment group in the optical axis direction. Moreover, the holding part 43 can be easily rotated around the central axis Cf.

In addition, as the configuration of the grip part 44 of the adjustment group, a configuration may also be employed in which a hole 66 is provided in a flange portion 65 as shown in FIG. 13. At least one hole 66 need only be provided around the periphery of the holding part 43. For example, in a case where a plurality of the holes 66 are provided, the holes 66 are spaced apart from each other in the circumferential direction Rf of the holding part 43 (refer to FIG. 12) not to communicate with each other.

The configuration of the grip part 44 of the adjustment group is not limited to the configuration in which the hole 66 is provided in the flange portion 65 as shown in FIG. 13, and may be a configuration in which the flange portion 65 is not provided and only the hole 66 is provided.

The hole 66 is, for example, circular, but the present invention is not limited to this, and the hole 66 may be polygonal or may be, for example, an ellipse extending along the circumferential direction Rf.

The present invention is basically configured as described above. Although the endoscope imaging device and the endoscope of the embodiment of the present invention have been described in detail above, the present invention is not limited to the above-described embodiments, and it is obvious that various improvements or modifications may be made within a range that does not depart from the gist of the present invention.

### EXPLANATION OF REFERENCES

10: endoscope system
12: endoscope
12a: distal end portion
14: light source device
16: processor device
18: distal end body
18a: distal end surface
18c: observation window
20, 20a: endoscope imaging device
21: first fixed group
22: first movement group
23: second movement group
24, 62: second fixed group
24a, 62a: adjustment group
25: sensor
25a: light-receiving surface
26: circuit board
26a: surface
27: prism
27a: incidence surface
27b: emission surface
27c: inclined surface
27e: reflecting surface
28: signal cable
28a: signal line
28b: covering layer
28c: shield conductor
28d: outer sheath
29: bump
30: lens barrel body
30a, 30b: end part
30c: end surface
31: lens storage part
32: housing
32a: end surface
32c: locking ring
32d: support portion
33: coupling part
34: first fixed lens
34a, 34b, 34c: lens
35: lens holding member
35a: holding portion
35b: flange
35c: storage portion
36: first movable lens
37: first movable lens holding part
37a: guide portion
37b: lens frame
37c: coupling portion
38: second movable lens
38a, 38b, 40a, 40b, 40c, 63a, 63b, 63c: lens
39: second movable lens holding part
39a: guide portion
39b: lens frame
39c: coupling portion
40, 63: second fixed lens
41: imaging lens
42: fixed lens holding member
43: holding part
43a: interior
43b, 43c: end part
43d: ring member
43e: side surface
43f: end part
43g, 43h: end surface
44: grip part
44a: flange portion
44b, 44c: recessed portion
45: lens drive portion
46: camshaft
46a: distal end
46b: flange
46c: end part
47: drive member
48: distal end support portion
49: cover glass
50: electronic component
60: movement group
64a, 64b: recessed portion
64c, 64d, 65: flange portion
64e: bottom surface
64f: bottom portion
66: hole
Lc: straight line
C: optical axis
CL, Cf, Ct: central axis
Cd: rotation axis
Dr: radial direction
Rf: circumferential direction
Rg: fitting portion
Sa, Sb: gap

What is claimed is:

1. An endoscope imaging device that acquires an image of an observation target, comprising:
at least one movement group including a movable lens that is movable along an optical axis;
a plurality of fixed groups including a fixed lens whose position is fixed with respect to the optical axis;
a sensor that receives light which has passed through the movable lens of the movement group and the fixed lens of the fixed group and that images the observation target;
a lens drive portion that moves the movement group along the optical axis; and
a lens barrel body provided with the movement group, the fixed group, and the lens drive portion,
wherein at least one of the plurality of fixed groups is an adjustment group,
the adjustment group includes a cylindrical holding part inside which the fixed lens is provided, the holding part includes a grip part provided at one end part of the holding part and an end surface provided at the other end part opposite to the one end part in an optical axis direction, and the grip part includes a projecting portion or a recessed portion whose diameter changes with respect to a radial direction of the holding part, and
the adjustment group is configured such that the end surface provided at the other end part of the holding part is disposed at a position spaced apart from the lens barrel body or the fixed group in the optical axis direction, and the grip part is disposed at a position spaced apart from the lens barrel body or the fixed group in the optical axis direction and protrudes from the lens barrel body or the fixed group, and a side surface of the holding part is fixed to the lens barrel body or the fixed group.

2. The endoscope imaging device according to claim 1,
wherein the adjustment group is provided at an end part of the lens barrel body on an observation target side or at an end part of the lens barrel body on a sensor side.

3. The endoscope imaging device according to claim 1,
wherein the grip part includes at least one projecting portion or recessed portion, or at least one hole, each of which is provided in a circumferential direction of the holding part, the at least one projecting portion or recessed portion having a diameter that changes with respect to the radial direction of the holding part.

4. The endoscope imaging device according to claim 2,
wherein the grip part includes at least one projecting portion or recessed portion, or at least one hole, each of which is provided in a circumferential direction of the holding part, the at least one projecting portion or recessed portion having a diameter that changes with respect to the radial direction of the holding part.

5. The endoscope imaging device according to claim 1,
wherein, among the plurality of fixed groups, a fixed group other than the adjustment group is mechanically positioned in the optical axis direction.

6. The endoscope imaging device according to claim 2,
wherein, among the plurality of fixed groups, a fixed group other than the adjustment group is mechanically positioned in the optical axis direction.

7. The endoscope imaging device according to claim 3,
wherein, among the plurality of fixed groups, a fixed group other than the adjustment group is mechanically positioned in the optical axis direction.

8. The endoscope imaging device according to claim 2,
wherein the fixed group is provided at the end part of the lens barrel body on the observation target side, and the adjustment group is provided at the end part of the lens barrel body on the sensor side.

9. The endoscope imaging device according to claim 4,
wherein the fixed group is provided at the end part of the lens barrel body on the observation target side, and the adjustment group is provided at the end part of the lens barrel body on the sensor side.

10. The endoscope imaging device according to claim 6,
wherein the fixed group is provided at the end part of the lens barrel body on the observation target side, and the adjustment group is provided at the end part of the lens barrel body on the sensor side.

11. An endoscope comprising:
the endoscope imaging device according to claim 1.

12. An endoscope comprising:
the endoscope imaging device according to claim 2.
